(19) United States
(12) Patent Application Publication
(10) Pub. No.: US 2008/0269162 A1
(43) Pub. Date: Oct. 30, 2008

(54) Novel Medicaments for Anti-Retroviral Treatments

(76) Inventors: Jean-Claude Yvin, Saint Malo (FR); Jean-Claude Chermann, Cassis (FR)

Correspondence Address: STITES & HARBISON PLLC, 1199 NORTH FAIRFAX STREET, SUITE 900, ALEXANDRIA, VA, 22314, US

(21) Appl. No.: 11/996,560

(22) PCT Filed: Apr. 13, 2007
(86) PCT No.: PCT/FR2007/000630

§ 371 Date: Jan. 23, 2008

(30) Foreign Application Priority Data
Apr. 14, 2006 (FR) 0603370

(51) Int. Cl. A61K 31/715; A61K 31/7072; A61P 31/14; A61P 31/18

(52) U.S. Cl. 514/50; 514/54

(57) ABSTRACT

This invention relates to the use of a sulphated or phosphated polysaccharide for the preparation of a medicine for treatments against retroviruses, more particularly against lentiviruses and oncoviruses, and particularly against HIV, including strains of these retroviruses that are resistant to known anti-retroviral agents; this medicine acts on the replication cycle of said retroviruses by inhibition of their RT.

The object of the invention is a novel medicament for treatments against retroviruses, acting on their replication cycle by inhibition of reverse transcriptase (RT).

Retroviruses belong to a family of viruses whose genome consists of RNA. The particular feature of the retroviruses is that they replicate in a host cell by passing through DNA stages, which is made possible by reverse transcriptase, or inverse transcriptase, designated by RT in the following, and which is an enzyme that enables transcription of the viral RNA into a molecule of complementary DNA called provirus.

The provirus is capable of annealing itself and integrating the genome of the host cell.

The retroviruses integrated in this fashion into the genome of the host cell can either use the cellular machinery to multiply itself or remain in a latent state in the host cell. In the latency state their genes are transmitted to the descendant cells with each mitosis and are temporarily silent; the organism carrying the retrovirus exhibiting no pathological sign.

The family of the retroviruses comprises three sub-families: the oncoviruses, the lentiviruses and the spumaviruses.

The oncoviruses are responsible for cancers, particularly for certain leukaemias. Of the group of oncoviruses causing cancer, the Roux sarcoma virus or RSV can be mentioned. Of those that cause leukaemias in the human being, there is the HTLV type I and type II (HTLV meaning <<Human T-cell Leukaemia Virus >>); in feline species, leukaemia is caused by the FTLV.

The lentiviruses are themselves responsible for slow-virus infections such as the acquired immune-deficiency syndrome or AIDS. In the human being, the lentiviruses responsible for AIDS are members of the HIV group or “Human Immunodeficiency Virus” (type I, HIV-I or type II, HIV-II); in the monkey it is the SIV (<<Simian Immunodeficiency Virus>>) and in the cat the FIV (<<Feline Immunodeficiency Virus >>).

Little is known about the spumaviruses both in terms of their structure and the exact manner of integration into the cellular genome. According to the current state of knowledge no disease appears to be associated with the spumaviruses. However, their involvement in triggering certain autoimmune diseases appears probable.

In terms of pathophysiology, the oncoviruses transform T-cells that are infected and bring about an uncontrolled proliferation of these cells the lentiviruses destroy the cells they infect.

As regards more particularly the HIV, these attack T4 helper lymphocytes that express on their surface the CD4 molecule (a membrane glycoprotein molecule), which is a receptor that enables the HIV to penetrate to the interior of the cell.

The HIV penetrate into the T4 lymphocyte by means of a system of endocytosis involving the mutual recognition and the binding of the expressed CD4 molecule on the surface of the T lymphocyte to a viral surface glycoprotein called gp120. Expression of a membrane co-receptor present on the T4 lymphocytes is also necessary to the penetration of the virus.

As indicated above, the retrovirus, here a HIV, replicates within the host cell, here the T4 lymphocyte, under the action of RT which gets involved in the manner described hereinbelow.

Initially, the nucleus or core of the virus, once it has penetrated to the interior of the host cell, releases two copies of single-stranded RNA.

These two copies of single-stranded RNA are associated with the RT as well as with other proteins such as protease and integrase.

The latter synthesizes a strand of complementary DNA from the viral RNA. Then, RNase activity associated with the RT degrades the RNA strand while a second strand of DNA is synthesized. The double-stranded DNA thus obtained is then annealed and then integrated, by virtue of an integrase enzyme, into the cellular genome to produce a provirus.

This provirus contains three structural genes, i.e. the gag (antigen group), pol (polymerase) and env (envelope); the gag genes code for the p24 viral proteins, the pol genes code for RT and its associated activities which are polymerase activities, RNase, integrase and protease, and the env genes code for the envelope glycoproteins such as gp120.

In the viral replication phase, the provirus is transcribed into messenger RNA by using the machinery of the host cell nucleus, which enables production of the constituent retroviral proteins on the one hand, and replication of the viral genomic material on the other hand. The assembly constitutes a new viral product by budding of the host cell membrane.

Furthermore, fusion phenomena are produced between infected cells, which present on there surfaces the gp120 protein, and non-infected T4 lymphocytes, which carry the CD4 molecule on their surfaces.

In fact, due to the high affinity of the gp 120 protein for the CD4 receptors, an infected cell can bind healthy non-infected T4 lymphocytes and form what is called a syncytium; in other words, an agglomerate of infected and non-infected lymphocytes, which is incapable of surviving.

One single infected cell can thus cause the death of numerous healthy T4 lymphocytes. A progressive decline of cellular immunity follows which translates into the development of opportunistic infections that may be accompanied by certain types of tumours.

In addition to the T4 lymphocytes, other cells also carry the CD4 molecule on their surfaces and are thus sensitive to the HIV virus; these are especially the macrophage monocytes, certain ganglionic cells, the skin and other organs and some B lymphocytes.

Moreover, the HIV virus can also attack certain cells of the central nervous system CNS. In this instance they cause neurological syndromes.

Other cell surface molecules that normally function as chimiokine receptors have also been identified as being co-receptors that enable entry of the HIV into the host cell.

The retrovirus responsible for AIDS, i.e. the lentiviruses and in particular HIV are characterized by extreme genetic and antigenic variability.

It is generally admitted that in the human being there are two types of virus responsible for AIDS that are designated, respectively, by HIV-I and HIV-II.

Genetic analyses have shown that there are three distinct groups of HIV-I; these three groups are designated by M (Major), O (Outlier) and N (New), respectively.

The great majority of HIV-I strains belong to the M (Major) group, which comprises at least ten sub-types or clades (A, B, C, D, . . . ); these clades are found in diverse geographical zones.

In contrast, HIV-I strains of the O or N groups have to date been isolated only in African populations.

The great genetic variability of these strains of the HIV is due principally to the elevated rate of RT errors. In fact, in the course of successive replication cycles, genetic variants appear.

The mutations are produced particularly in the env gene and more precisely in the part coding for gp120.

This genetic variability translates into the appearance of strains that become resistant to known anti-retroviral agents.

The treatments currently used to combat these retroviruses, particularly the lentiviruses and more particularly HIV, are aimed at inhibiting RT and consequently of blocking its replication cycle.

It must be noted that in the case of the oncoviruses associated with certain types of cancer, RT is always essential to their replication. In addition, when this viral enzyme is inhibited, the oncovirus cannot replicate itself anymore in the host cell.

Current anti-retroviral treatments implement three major therapeutic groups: The reverse transcriptase (RT) inhibitors, the protease inhibitors and the fusion and entry inhibitors.

Among the inhibitors of reverse transcriptase, three sub-groups of molecules are actually used clinically. On the one hand these are the nucleoside inhibitors of RT; on the other hand the non-nucleoside inhibitors of RT and finally the nucleotide analogues.

Of the nucleoside inhibitors of RT AZT (or 3′-azidothymidine), diagnosing and stavudine (d4T, Zérit, which are analogues of thymidine) and ddC and 3TC (which are analogues of cytidine) may be mentioned. These nucleoside inhibitors enter into competition with the natural nucleosides and prevent extension of the DNA chain; they were the first to be used as RT inhibitors initially alone and then in combination with other RT inhibitors or viral enzymes like protease and integrase. Therefore, their undesirable effects are well known and numerous. In particular, the mutations of reverse transcriptase confer a resistance to NIRTs, which can be crossed among several NIRTs. The compounds are all neutral or reducers, with the exception of AZT, which is an oxidant;

The non-nucleoside inhibitors of RT act as non-competitive antagonists by binding to a hydrophobic region adjacent to the catalytic site of RT, thus inhibiting the latter; among them ritonavir, saquinavir, efavirenz, rescriptor, sustiva and viramune may be mentioned.

The second therapeutic group consists of the protease inhibitors (PI). These are powerful anti-retroviral agents that inhibit the proteolytic activity of viral protease; amprenavir, tipranavir, indinavir, saquinavir, lopinavir, posanprenavir, ritonavir, atazanavir and nelfinavir may be mentioned by way of example.

Finally, the third therapeutic group corresponds to the fusion and entry inhibitors, of which several molecules are being studied. Only enfuvirtide is currently on the market. It acts on the first stage of viral replication by competitive inhibition preventing virus/cell fusion.

The appearance and transmission, particularly in the industrialized countries, of HIV strains that are resistant to known anti-viral agents has confronted the medical world with a serious public health problem.

The treatment failures encountered in patients with AIDS are actually due mainly to phenomena of viral resistance, although other factors such as the toxicity of the agents used and certain secondary effects also influence the efficacy of the treatments even if this is to a lesser degree.

Numerous research studies have been conducted to meet the challenge of this situation and to discover new anti-retroviral agents capable of inhibiting RT, particularly among the polysaccharides.

European patent application 0 240 098 discloses the use of synthetic polysaccharides or polysaccharides naturally sulphated by means of connector groups as anti-retroviral agents. EP 0240 098 discloses in particular sulphates of chondrotoin, dermatan, keratan, hyaluronic acid, carrageenan, fucoidan, heparin and dextran.

Patent application EP 0 464 759 also described polysaccharides sulphated by means of a specific group and intended for long-term prophylaxis of diseases caused by viruses.

These two patent applications, EP 0 240 098 and EP 0 464 759, present the major drawback of proposing complex polysaccharides synthesized by the presence of an intermediary group necessary for sulphation.

The Japanese patent application published under No. 01-103 601 describes the anti-viral activity, in particular with regard to HIV, of lentinan sulphate, certain β-1,3 glucans such as curdlan, panhuman and those of the cell walls of yeasts as well as those of cellulose.

In an article that appeared in November 1987 in the Jpn. J. Cancer Res/Gann No. 78, pp. 1164-1168, Hideki Nakajima et al. describe the inhibitory effect on the infectivity and replication of HIV of certain sulphated polysaccharides such as the sulphates of dextran, xylofuranan and ribofuranan as well as the inhibition by these products of HIV RT.

Japanese patent application published under No. 03-145 425 describes the antiviral activity with regard to HIV of certain sulphated laminarioligosaccharides and in particular of sulphated laminaripentaose.

The publications described in these citations, however, have not resulted in any practical application, particularly due to the fact that the oligosaccharides described, even though they have potential anti-retroviral activity, have a powerful anticoagulant activity such as the sulphates of dextran and heparin, for example, which renders them useless in vivo in the context of therapy.

The problem addressed by the present invention is that of providing the field of medicine with novel anti-retroviral medicaments at higher therapeutic index, particularly active against the lentiviruses and the oncoviruses, especially against HIV, as well as against strains resistant to certain anti-retroviruses already known and having low anti-coagulant activity in vivo.

In a surprising and unexpected way, this problem was resolved by the applicant, who has determined that some sulphated or phosphated polysaccharides having the formula (I) as described hereinafter, have powerful anti-retroviral activity, particularly against the lentiviruses and oncoviruses, particularly against HIV and against the retroviral strains resistant to already known anti-retrovirus agents inhibiting RT, and do not have anticoagulant activity incompatible with in vivo administration.

Thus, an object of the present invention is the use, for the manufacture of a medicament for the treatment of retroviral diseases, of a polysaccharide having the formula (I)

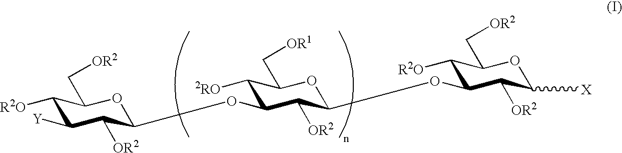

wherein

R¹ represents either a hydrogen atom, a sulphate group or a phosphate group, or a sulphated or phosphated glucose preferably linked by a β(1→6) type link to the saccharide structure,

R² represents a hydrogen atom, a sulphate group or a phosphate group, provided that R¹ and R² do not represent simultaneously a hydrogen atom,

X and Y represent, each independently, an OH group, a glucose, a sulphated or phosphated glucose, a mannitol or a sulphated or phosphated mannitol,

n represents an integer from 11 to 30, preferably from 20 to 30 and more preferably from 25 to 30,

said polysaccharide having a sulphation degree greater than 2, preferably from 2.2 to 2.4, or a phosphation degree greater than 1, preferably from 1.5 to 2.5.

According to another embodiment, the polysaccharide used is a polysaccharide of formula (I) wherein R₁ and R₂ can be identical and then represent a sulphate or phosphate group, or different from each other, R₁ then representing a sulphated or phosphated glucose unit linked preferably by a β-1,6 type β link to the saccharide structure, X and/or Y representing a mannitol group and n an integer from 11 to 30, more particularly from 25 to 30.

According to the invention, the medicament manufactured by the use of a polysaccharide having the formula (I) acts on the replication cycle of the retrovirus by inhibiting the RT of same.

In the meaning of the present invention, “sulphation degree” is defined as the mean number per saccharide unit of sulphated OH groups. A sulphation degree greater than 2 means that, on average, over the entire polysaccharide, more than 2 OH groups per saccharide unit are sulphated.

Within the meaning of the present invention, “phosphation degree” is defined as the mean number per saccharide unit of phosphated OH groups. A phosphation degree greater than 1 means that, on average, over the entire polysaccharide, more than 1 OH group per saccharide unit is phosphated.

In the meaning of the invention, “sulphate group” is defined as a group of the type (—SO₃H).

In the meaning of the invention, “phosphate group” is defined as a group of the type (—PO₃H₂).

A further object of the invention is the use of one of the aforementioned polysaccharides for implementing a method of treatment of retroviral diseases.

Within the meaning of the invention, the retroviral diseases are selected preferably from the group caused by the lentiviruses and the oncoviruses, more particularly by HIV and by the strains of these retroviruses that are resistant to the already known anti-retroviral inhibitors of RT. In the case of the lentiviruses and in particular of the type I and II HIV, the medicament obtained according to the invention by the use of a polysaccharide having the formula (I) enables the treatment of the acquired immunodeficiency syndrome in the human being. Thus, in one particular embodiment a retroviral disease in the meaning of the invention is the acquired immunodeficiency syndrome or AIDS in the human being.

The polysaccharides of formula (I) as used in accordance with the invention are also particularly active against the oncoviruses and in particular against HTLV type II and I. Thus, in one particular embodiment, the use according to the invention of a polysaccharide having the formula (I) enables the treatment of cancers associated with these retroviruses.

In one particular embodiment of the invention, the polysaccharide having the formula (I) is a sulphated laminarin having a polymerisation degree of 11 to 28.

Preferably, the polysaccharide having formula (I) is a laminarin having a sulphation degree equal to around 2.3 and called “laminarin PS3”.

Within the meaning of the invention, “polymerisation degree” is defined as the number of monosaccharide units linked to each other by β(1→3) type links comprising the main linear chain. A polymerisation degree of 11 to 28 means a polysaccharide consisting of 11 to 28 saccharide units, e.g. glucose, linked to each other by β(1→3) type linkages. This polymerisation degree does not take into account glucose units β(1→6) linked to the main chain of the polysaccharide. Thus, the polymerisation degree is equal to n+2 when X and Y simultaneously represent OH, to n+3 if only one of X or Y represents OH and to n+4 if neither X nor Y represents OH.

In an unexpected and surprising manner, the applicant demonstrated that the sulphated laminarin, having a sulphation degree greater than 2, preferably from 2.2 to 2.4, and a polymerisation degree of 11 to 28, was particularly efficient in the treatment of retroviral diseases, preferably selected from those caused by the lentiviruses and the oncoviruses, more particularly by HIV and by the strains of these retroviruses that are resistant to the anti-retroviral inhibitors of RT. In addition, said sulphated laminarin has low anticoagulant activity, thus confirming its great interest for manufacturing a medicament intended for administration to human beings or to animals.

According to another embodiment of the invention, the invention relates to the use of a polysaccharide obtained using sulphated laminarin having a sulphation degree greater than 2 and preferably from 2.2 to 2.4, a polymerisation degree of 11 to 28, for the manufacture of a medicament for the treatment of retroviral diseases, preferably selected from those caused by the lentiviruses and the oncoviruses and more particularly by HIV, and by the strains of these retroviruses that are resistant to the already known anti-retroviral inhibitors of RT.

In another particular embodiment of the invention, the polysaccharide having the formula (I) is a sulphated laminarin having a polymerisation degree of 11 to 28. The phosphate of laminarin according to the invention has a phosphation degree greater than 1 and preferably of 1.5 to 2.5 and is particularly adapted to the treatment of retroviral diseases, preferably selected from among those caused by the lentiviruses and the oncoviruses, more particularly by HIV, and by the strains of these retroviruses that are resistant to the already known anti-retroviral agents inhibiting RT.

According to another embodiment of the invention, the invention relates to the use of a polysaccharide obtained from sulphated laminarin having a sulphation degree greater than 1 and preferably from 1.5 to 2.5, a polymerisation degree of 11 to 28, for the manufacture of a medicament for the treatment of retroviral diseases, preferably selected from those caused by the lentiviruses and the oncoviruses and more particularly by HIV, and by the strains of these retroviruses that are resistant to the already known anti-retroviral inhibitors of RT.

One particular embodiment of the invention relates to the use of a sulphated laminarin characterized in that it has a sulphation degree that is greater than 2, preferably from 2.2 to 2.4 and a polymerisation degree of 11 to 28 for the manufacture of a medicament for the treatment of retroviral diseases.

Another particular embodiment of the invention relates to the use of a sulphated laminarin characterized in that it has a sulphation degree that is greater than 2, preferably from 2.2 to 2.4 and a polymerisation degree of 11 to 28 for implementing a method of treatment of retroviral diseases.

Another particular embodiment of the invention relates to the use of a phosphated laminarin characterized in that it has a phosphation degree of greater than 1, preferably from 1.5 to 2.5 and a polymerisation degree of 11 to 28, for the manufacture of a medicament for the treatment of retroviral diseases.

Yet another particular embodiment of the invention relates to the use of a phosphated laminarin characterized in that it has a phosphation degree of greater than 1, preferably from 1.5 to 2.5 and a polymerisation degree of 11 to 28, for implementing a method of treatment retroviral diseases.

It is well known to administer to patients suffering notably from AIDS, in the context of treatments designated by the term “combination therapy”, not only two or three antiretroviral agents but also other pharmacological agents to treat associated pathologies. The take of these numerous medicaments is a considerable task for the patients. In order to circumvent this drawback and according to another advantageous embodiment, the invention also relates to a combination product comprising an effective amount of:

a polysaccharide having the formula (I) wherein R₁ represents either a hydrogen atom, a sulphate group or a phosphate group or a sulphated or phosphated glucose linked, preferably by a β(1→6) type linkage to the saccharide structure, R₂ represents a hydrogen atom, a sulphate group or a phosphate group, X and Y each independently represent an OH group, a glucose, a sulphated or phosphated glucose, a mannitol, or a sulphated or phosphated mannitol, n represents an integer from 11 to 30, preferably from 20 to 30, more preferably from 25 to 30, said polysaccharide having a sulphation degree greater than 2, preferably from 2.2 to 2.4, or a phosphation degree greater than 1, preferably from 1.5 to 2.5,

of at least one anti-retroviral agent selected from the group comprising:

nucleoside inhibitors of reverse transcriptase (NIRT), notably AZT, ddl, ddC, d4T, 3TC and ABC,

the non-nucleoside inhibitors of reverse transcriptase (NNIRT), notably Viramune and Sustiva,

the protease inhibitors, notably Agnerase and Kaletra,

the fusion inhibitors, notably enfuvirtide (Fuzeon),

the entry inhibitors, notably AMD-3100 and, optionally

at least one pharmacological agent selected from the group comprising the anti-nausea agents, the anti-diarrhoea agents, the anti-hyperbilirubinemia agents, the analgesic agents, the dermatological treatment agents, the anti-nephrotoxic agents, for simultaneous, separate or stepped use over time.

In fact, the specialist in the art must also define the most appropriate administration, making possible to obtain the best therapeutic index for the patient. Each active substance used in the combination therapy can be administered sequentially, or by different routes, or even at the same time.

Within the meaning of the invention, “effective amount” is defined as a quantity of active substance that is sufficient to obtain a therapeutic effect in a patient.

A further object of the invention is the use of an effective amount

of a polysaccharide having the formula (I) as described hereinbefore;

of at least one anti-retroviral agent selected from the group comprising:

nucleoside inhibitors of reverse transcriptase (NIRT), notably AZT, ddl, ddC, d4T, 3TC and ABC,

the non-nucleoside inhibitors of reverse transcriptase (NNIRT), notably Viramune and Sustiva,

the protease inhibitors, notably Agnerase and Kaletra,

the fusion inhibitors, notably enfuvirtide (Fuzeon),

the entry inhibitors, notably AMD-3100 and, optionally

at least one pharmacological agent selected from the group comprising the anti-nausea agents, the anti-diarrhoea agents, the anti-hyperbilirubinemia agents, the analgesic agents, the dermatological treatment agents, the anti-nephrotoxic agents, for the manufacture of a medicament for the treatment of retroviral diseases, preferably those caused by the lentiviruses and the oncoviruses, more preferably caused by HIV, particularly by the strains of these viruses that are resistant to the already known anti-retroviral agents.

A further object of the present invention is the use of an effective amount

of a polysaccharide having the formula (I) as described hereinbefore;

of at least one anti-retroviral agent selected from the group comprising:

nucleoside inhibitors of reverse transcriptase (NIRT), notably AZT, ddl, ddC, d4T, 3TC and ABC,

the non-nucleoside inhibitors of reverse transcriptase (NNIRT), notably Viramune and Sustiva,

the protease inhibitors, notably Agnerase and Kaletra,

the fusion inhibitors, notably enfuvirtide (Fuzeon),

the entry inhibitors, notably AMD-3100 and, optionally

at least one pharmacological agent selected from the group comprising the anti-nausea agents, the anti-diarrhoea agents, the anti-hyperbilirubinemia agents, the analgesic agents, the dermatological treatment agents, the anti-nephrotoxic agents,

for implementing a method of treatment retroviral diseases, preferably those caused by the lentiviruses and the oncoviruses, more preferably caused by HIV, particularly by the strains of these viruses that are resistant to the already known anti-retroviral agents.

The present invention also relates to a method of treatment of a retroviral disease, preferably caused by the lentiviruses and the oncoviruses, more preferably by HIV, especially by the strains of these retroviruses that are resistant to the already known anti-retroviral agents, comprising in the administration to a patient with said retroviral disease of an effective amount of a medicament comprising as its active agent at least one polysaccharide having formula (I) as described hereinbefore; or of a combination product as described hereinbefore.

In the meaning of the present invention, “patient” is defined as any warm-blooded animal, particularly mammals and especially human beings.

A further object of the present invention is a method of treatment as defined hereinbefore and wherein the polysaccharide having formula (I) is a sulphated laminarin having a sulphation degree greater than 2, preferably of 2.2. to 2.4, and a polymerisation degree of 11 to 28.

A further object of the present invention is a method of treatment as defined hereinbefore and wherein the polysaccharide having formula (I) is a phosphated laminarin having a phosphation degree greater than 1, preferably of 1.5 to 2.5, and a polymerisation degree of 11 to 28.

To prepare a sulphated polysaccharide of formula (I) according to the invention, a sulphation stage is carried out preferably in accordance with the protocol described by Alban S., Kraus J., and Franz G. in “Synthesis of laminarin sulphates with anticoagulant activity”, Artzneim. Forsch./drug Res (1992) 42; 1005-1008. This process was improved in the thesis by Susanne Alban, defended in 1993 at the University of Regensburg and entitled “Synthese und physiologische Testung neuartiger Heparinoide”. These methods can be adapted to the sulphation of polysaccharides having formula (I) of the invention and make it possible to easily obtain in a cheap manner a highly substituted sulphated polysaccharide without degradation and with good reproducibility.

In order to achieve effective sulphation of the polysaccharide without degradation of the polysaccharide chains, the sulphation reaction is advantageously done under conditions equivalent to an absolute absence of water. Before sulphation, the polysaccharide is preferably dry, e.g. over phosphorus pentoxide (P₂O₅), and then dissolved in dimethyl formamide or DMF. In virtue of its alternative effects on the polysaccharide, the DMF has an activating influence through the substitution. In fact, the association of the polar DMT with the OH groups results in the cleaving of the inter- and intramolecular hydrogen bonds and in the disintegration of the higher structures.

To implement the sulphation reaction, one could advantageously use the SO₃ pyridine complex.

As a result of the coordination of SO₃ electron acceptor with the pyridine electron donor, the difficulty to control the reactivity of the SO₃-which is expressed by a strongly exothermic reaction involving degradations is reduced. The SO₃-pyridine complex has in comparison with other complexes the advantage of not being excessively reactive nor excessively stable, in other words too slow from the point of view of reaction.

By reason of the fact that the sulphation degree which is obtained is proportional to the molar excess of sulphation reagent and given that it is sought to obtain a sulphation degree greater than 2, a concentration of 6 moles of SO₃-pyridine per mole of glucose is used advantageously.

Advantageously, in order to ensure the absence of water, one can work under an argon atmosphere.

Preferably, from the start of the reaction, pyridine can be added to the sulphation reagent in equimolar quantities, in view of directly capturing the sulphuric acid that could be formed by the reaction of the SO₃-pyridine complex with water. The concentration of the polysaccharide and that of the sulphation reagent must be preferably as high as possible, since the solubility of the polysaccharide and of the sulphation reagent limit the final sulphation degree. In order to prevent cooling of the mixture at the start of the reaction that could result in solubility problems and in order to obtain a substitution that is as regular as possible, the solution of the SO₃-pyridine complex in the DMF may not be added at once but continuously over a period of 4 hours.

The sulphation reaction may be done at a temperature of 20 to 60° C., preferably around 40° C. Higher temperatures result in a more efficient substitution but also in a degradation of the chains.

After the addition of the sulphation reagent, the mixture is preferably agitated for several hours at around 60° C. At this temperature, a supplementary substitution is produced without degradation of the chains.

The supernatant of the mixture is then advantageously separated by decantation. The residue is dissolved, preferably in NaOH, then mixed with 10× its volume of ethanol. The precipitate that is produced at a temperature of 4-8° C. overnight is isolated then prderably dissolved in diluted sodium hydroxide (pH of the solution about 9). The solution is dialysed in order to remove the salts and low molecular weight molecules and then advantageously brought to a pH of 7.0 by the addition of NaOH and then lyophilised. The resulting sulphated polysaccharide is present in the form of a sodium salt.

The sulphation degree is preferably determined using conductimetric titration of free acid of the sulphated polysaccharide or alternatively by ionic chromatography after hydrolysis using a HPLC system. The first method has the advantage of being also suitable to studies relating to stability (the consumption of sodium hydroxide increases when the sulphates groups are eliminated) while the HPLC method requires less substance and can be automated. By way of control, it is possible to determine the sulphur content using elementary analysis.

It is further possible to control the homogeneity of sulphation and the distribution of the sulphates groups on the different positions in the glucose molecule using a modified form of analysis of methylation followed by a GC-MS assay (i.e. gas chromatography, mass spectrometry).

The sulphation degree obtained in proceeding as indicated hereinbefore is greater than 2, more precisely 2 to 2.5 and quite particularly from 2.2 to 2.4.

According to one advantageous embodiment, the polysaccharide having formula (I), and preferably the sulphated laminarin, are used for the manufacture of a medicament for the treatments against retroviruses and intended for general administration and preferably by oral, rectal, pulmonary, topical (including transdermal, buccal and sublingual) and parenteral (including sub-cutaneous, intramuscular, intravenous, intradermal and intravitreal) administration.

The daily dose is generally of 0.01 to 250 mg per kilogram of patient weight and preferably of 0.10 to 100 mg, more preferably of 0.5 to 30 mg and most preferably of 1.0 to 20 mg.

These daily doses apply especially in the case of laminarin sulphate; for the other salts and esters according to formula (I), the daily doses are adapted on a case-by-case basis.

The daily dose can be administered by unit dose in one, two, three, four, five or six times or more at different times of the day.

The unit doses can be from 10 to 1000 mg, from 50 to 400 mg and, preferably, from 50 to 100 mg of active substance.

The medicaments obtained according to the invention, by using at least one of the polysaccharides having formula (I), comprise classical formulation ingredients and optionally one or a plurality of other therapeutic agents.

Furthermore, as mentioned hereinabove, the inventive polysaccharide can be advantageously combined with other active substances. Their mode of administration can be simultaneous or sequential. The can also be administered by different routes as described hereinabove.

The present invention will be better understood by reading the non-limiting examples that follow.

### EXAMPLE 1

### Preparation of a Laminarin Sulphates PS3

The laminarin is extracted from a starting material consisting of brown algae, then laminarin is sulphated then extracted by following the protocol described in French patent No. 92 08387.

Once the laminarin is extracted and sulphation was done according to the protocol described by Alban S., Kraus J., and Franz G. in “Synthesis of laminarin sulphates with anticoagulant activity”, Artzneim. Forsch./drug Res. (1992) 42; 1005-1008, perfected by the thesis by Susanne Alban, defended in 1993 at the University of Regensburg and entitled “Synthesis and Physiological Testing of Novel Heparinoids”.

Initially the laminarin was dried over phosphorus pentoxide (P₂O₅) then dissolved in dimethyl formamide or DMF.

Then, in order to start the sulphation reaction, the SO₃-pyridine complex was used under argon: SO₃-pyridine was added to the DMF in equimolar quantities only once but in continuous manner over a period of 4 hours. The sulphation reaction was carried out at a temperature of 40° C. After the addition of the sulphation reagent, the mixture was continuously stirred over 6 hours at 60° C.

The supernatant was then separated from the mixture by decantation, the residue dissolved in 2.5 M NaOH and then the mixture was combined with 10-times its volume of 99% ethanol. The solution obtained in this process was then placed at a temperature of 4-8° C. overnight and a precipitate obtained that was isolated and then dissolved in dilute sodium hydroxide (pH of the solution about 9). The solution was then dialyzed using a Spectrapor membrane with a cut off of 1000 D then brought to pH 7.0 by the addition of NaOH. Finally, the dialyzed solution was lyophilised. A laminarin sulphate in the form of a sodium salt was obtained.

The sulphation degree was then determined by means of conductimetric titration of free acid of the sulphated polysaccharide by using 0.1N sodium hydroxide. The sulphation degree of the laminarin obtained was 2.3. The polymerisation degree of the laminarin sulphates thus obtained was 23 to 28.

This sulphated polysaccharide was named “laminarin PS3” or otherwise “PS3”.

### EXAMPLE 2

### Anti-Retroviral Activity of the Sulphated Laminarin PS3

The action of the sulphated laminarin PS3 on the retroviral replication cycle by RT inhibition was determined; said inhibition of RT was appreciated

either by the observation, as a function of the quantity of sulphated polysaccharide used and of the time of said use, which can be made before, during or after infection, or even continuously, and of the reduction of the number of syncitia appearing in a culture of MT₄ cells following infection by a HIV retrovirus (example 2A),

or by appreciation as a function of the quantity of sulphated polysaccharides used and of the time of said use, which can be made before, during or after the infection or even continuously throughout the infection, of inhibition of RT that results in a measurable reduction of the activity of said RT, and which is

reflected in a slowing of infecting viral replication in a culture of

CEM cells infected by an HIV retrovirus (Example 2B), it being understood that by way of comparison, the same observations were done using as comparison products on the one hand dextran sulphate and on the other hand 3′-azidothymidine or AZT.

The aforementioned MT₄ and CEM cells are human HTLV-1 transformed lymphoblast lines.

The retroviruses that were used consist of

BRU (clade B) and NDK (clade D) laboratory strains;

the RTMC (clade B) virus known to be resistant to the antiviral agent AZT;

RW92009 (clade A) and UG92029 (clade A) primary isolates, and

an isolate (PIC CH, clade B) obtained from a patient who was resistant to several known antiviral agents such as d4T and Zerit.

The RTMC, RW92009 and UG92029 strains were obtained from the “AIDS Research and Reference Reagent Program, Division of AIDS, NIAID-NIH (USA)”.

The MT₄ and CEM cells were cultured in RPMI medium (Cambrex) supplemented with 10% foetal calf serum (Cambrex), 1% penicillin-streptomycin (Life Technologies), 2 mM of glutamine (Invitrogen), 2 μg/mL of polybrene (Sigma).

Lyophilised sulphated laminarin PS3 obtained by the process described in Example 1 was used, and dissolved in PBS at a concentration of 12 mg/mL in order to obtain a mother solution.

On the other hand, detran sulphate (Sigma, D4911) was dissolved in PBS to a concentration of 41.6 mg/mL (mother solution).

AZT (Sigma A2169), widely known to be a RT inhibitor, was likewise diluted in PBS to the concentrations indicated hereinafter.

### EXAMPLE 2A

### Measurement of the Number of Syncitia

The action of laminarin PS3 and that of the comparison products was evaluated using the number of syncitia in a culture of MT₄ cells infected by one of the aforementioned viruses by proceeding as described below.

First of all MT₄ cells (3*10⁵ cells in 50 μL) were preincubated for 1 hour at 37° C. in a wet 5% CO₂ enriched atmosphere with serial dilutions of sulphated laminarin PS3 (50 μL) and comparison products in a 96-well V-bottom microplate. Each concentration of sulphated laminarin PS3 and the comparison product was tested in duplicate.

Then infection of the cells was carried out by adding to each well, with the exception of those wells being used as control cells, 50 μL of previously titrated virus dilution.

After an additional hour of incubation, the plate was centrifuged and the supernatant containing the residual virus was discarded.

Two rinses were done; then the cell pellets were transferred to a 24-well plate where the cells were cultured at a concentration of 3*10⁵ cells/mL in the presence of sulphated laminarin PS3 or a comparison product.

After 3 days of culture, the cells were diluted to half and the syncitia were observed using the inverse microscope from day 3 to day 7 after having been placed in suspension.

As already stated above, four series of experiments were done with the intention of determining the effect of the sulphated laminarin PS3 as a function of the time at which it was incorporated in the cell culture; that is to say

before infection

during infection

after infection, and

throughout the infection.

In the first case, the sulphated laminarin PS3 and the comparison products were brought in contact with the cells for one hour, then the cells were washed twice in order to eliminate the products used and then the MT₄ cell cultures were infected.

In the second case, the sulphated laminarin PS3 and the comparison products were brought in contact at the moment at which the cells were infected. The culture treated in this manner was kept intact for one hour and then washed twice, which resulted in the elimination of the sulphated laminarin PS3 and of the comparison products as well as the viruses that did not penetrate into the cells.

In the third case, the MT₄ cell culture was preincubated for one hour at 37° C., then the cells were infected, the whole was subjected to incubation for one hour at 37° C., then to centrifugation with the elimination of the supernatant containing the virus that had not penetrated into the cells. The cell pellet was then rinsed twice and then transferred to a 24-well plate, and then cultured at a concentration of 3*10⁵ cells/mL simultaneously with the introduction into the same wells of the sulphated laminarin PS3 and the comparison products at the selected concentrations.

In the fourth case, we started by putting the sulphated laminarin PS3 and the comparison products in contact with the cells for one hour at 37° C., then the culture was infected for one hour a 37° C. Then the cultures were washed twice, which resulted in the elimination of the virus that had not penetrated into the cells. The cell pellets were then transferred to 24-well plates at a rate of 3*10⁵ cells/mL by well in the presence of the sulphated laminarin PS3 and the comparison products at the chosen concentrations.

The washings and rinsings were done using RPMI without foetal calf serum.

In each of the four types of experiments that have just been described, and as previously indicated, the cells were diluted to half after three days of culture and then the syncitia were observed using the inverted microscope from day 3 to day 7.

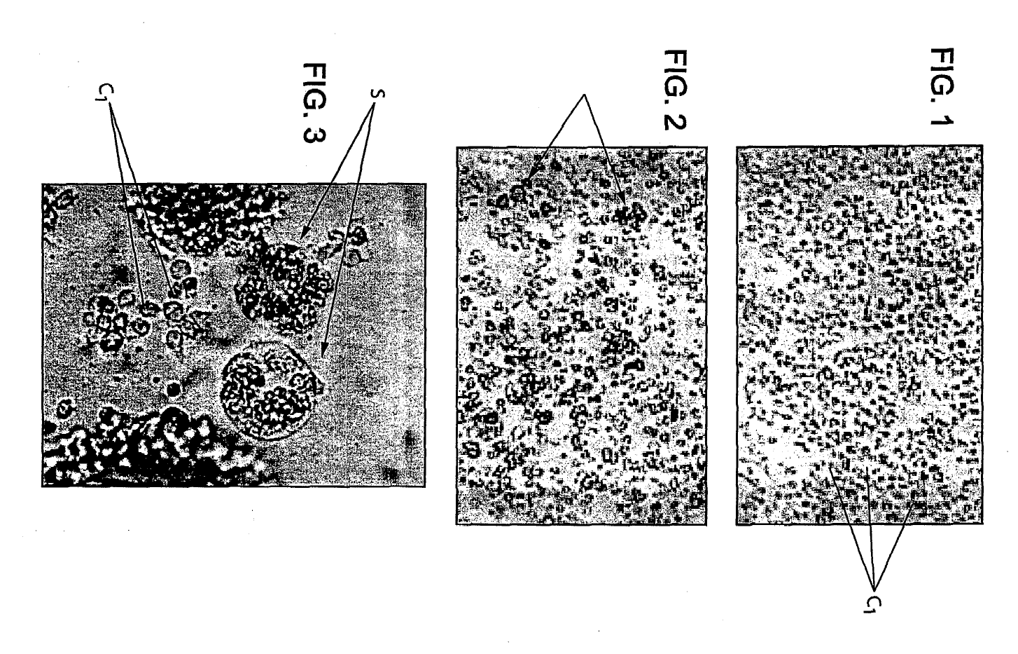

The results of these observations are shown in FIGS. 1 to 3, wherein

FIG. 1 shows a culture of uninfected C₁ cells; the C₁ cells remain distinct from each other; they have no syncitia;

FIG. 2 shows a culture of infected cells in which certain C₁ cells agglutinate into syncitia (marked with S), and

FIG. 3 shows, at higher magnification, a culture of infected C₁ cells, certain of the C₁ cells being agglutinated into syncitia (marked with S).

In the experiments described in greater detail hereinafter, the absence of syncitia is indicated by a minus (−) sign and the (+), +, ++, ++T signs indicate an increasing number of syncitia in each well.

The indication “T” indicates the death of the cells.

In a first group of experiments, AZT was used as the comparison product at a concentration of 0.01 μm, and sulphated laminarin PS3 was incorporated at two concentrations, 5 and 10 μg/mL, respectively.

The BRU virus (clade B) was used for infecting the MT₄ cells at a viral dilution of 10⁵, which corresponds to the quantity of viral particles capable of infecting 80% of the MT₄ cultures (Tissue Culture Infections Dose 80%, TCID_{80%}).

The results observed from day 3 to day 4 in the four types of experiments defined hereinbefore are summarized in Table A.

TABLE A

| Anti-retroviral Agent | | | | Number of Syncitia in the | | | | |
|---|---|---|---|---|---|---|---|---|
| Incor- | | | | Culture (wells) at Days 3 to 7 | | | | |
| poration | Nature | Concentration | | J3 | J4 | J5 | J6 | J7 |
| Continuous, | PS3 | 10 | μg/mL | − | − | − | − | + |
| before, | | | | − | − | − | − | − |
| during and | | 5 | μg/mL | − | − | − | (+) | + |
| after | | | | − | − | (+) | (+) | ++ |
| infection | AZT | 0.01 | μM | − | − | − | − | − |
| | | | | − | − | − | − | − |
| Before | PS3 | 10 | μg/mL | − | (+)/+ | ++ | ++T | ++T |
| infection | | | | − | − | +/++ | ++ | ++T |
| | | 5 | μg/mL | − | + | +/++ | ++T | ++T |
| | | | | − | (+)/+ | ++ | ++T | ++T |
| | AZT | 0.01 | μM | − | (+) | ++ | ++T | ++T |
| | | | | − | (+) | + | ++ | ++T |
| During | PS3 | 10 | μg/mL | − | (+) | + | ++ | ++T |
| infection | | | | − | (+) | + | ++ | ++T |
| | | 5 | μg/mL | (+) | + | ++ | ++ | ++T |
| | | | | (+) | + | ++ | ++ | ++T |
| | AZT | 0.01 | μM | − | (+) | + | ++ | ++T |
| | | | | − | (+)/− | + | ++ | ++T |
| After | PS3 | 10 | μg/mL | − | − | − | − | − |
| infection | | | | − | − | − | − | − |
| | | 5 | μg/mL | − | − | − | − | − |
| | | | | − | − | − | − | − |
| | AZT | 0.01 | μM | − | − | − | − | − |
| | | | | − | − | − | − | − |

| | | | | | |
|---|---|---|---|---|---|
| Absence of anti-retroviral agent | − | (+) | + | ++ | ++T |
| | − | (+) | +/++ | ++ | ++T |
| Absence of virus | − | − | − | − | − |
| Absence of anti-retroviral agent | − | − | − | − | − |

The results summarized in Table A make it possible to make the statements that follow.

After 6 days counting from the infection by the BRU viral strain, the cultures of MT₄ do not form syncitia when they are treated continuously from their placement in culture (before, during and after infection), using concentrations of 10 μg/mL of PS3.

The MT₄ cell cultures do not form syncitia when they are treated with concentrations of 10 and 5 μg/mL of PS3 after infection by the BRU viral strain. The effect is observed from the 3^{rd} day after viral infection.

It is important to note that no inhibitory effect was observed when the MT₄ cells are treated before or during infection by the viral strain.

It can also be seen in Table A that PS3 is as efficient as AZT.

In view of these results, it appears that in the case of PS3, it is a specific effect on the replication of the virus and not an non-specific effect due to the anionic nature of PS3 on the entry of the virus into the cell (there is no action when the PS3 is placed in contact only before infection).

In a second group of experiments, AZT and dextran sulphate were used as comparison products and incorporated, respectively, at concentrations of 0.4 μM and 10 μg/mL, this latter concentration also being that of the PS3.

The NDK virus (clade B) was used for infecting the MT₄ cells at a viral dilution of 2.5*10⁵, which corresponds to the quantity of viral particles capable of infecting 80% of the MT₄ cultures (Tissue Culture Infections Dose 80%, TCID_{80%}).

The results observed from day 3 to day 6 are summarized in Table B.

TABLE B

| | | | | Number of Syncitia | | | |
|---|---|---|---|---|---|---|---|
| Anti-retroviral Agent | | | | in the Culture (wells) | | | |
| | | Final | | at Days 3 to 6 | | | |
| Incorporation | Nature | concentration | | J3 | J4 | J5 | J6 |
| Before infection | PS3 | 10 | μg/mL | − | + | ++ | T |
| | | | | − | (+) | + | T |
| | Dextran | 10 | μg/mL | − | − | − | − |
| | (sulphates) | | | − | − | − | − |
| | AZT | 0.4 | μg/mL | − | + | ++ | T |
| | | | | − | − | − | (+) |
| During infection | PS3 | 10 | μg/mL | − | − | − | − |
| | | | | − | − | − | − |
| | Dextran | 10 | μg/mL | − | − | − | ++ |
| | (sulphate) | | | − | (+) | ++ | T |
| | AZT | 0.4 | μm | − | − | − | − |
| | | | | − | − | − | − |
| After infection | PS3 | 10 | μg/mL | − | − | − | − |
| | | | | − | − | − | − |
| | Dextran | 10 | μg/mL | − | − | − | − |
| | (sulphate) | | | − | + | ++ | T |
| | AZT | 0.4 | μm | − | − | − | − |
| | | | | − | − | − | − |
| Continuous, before, | PS3 | 10 | μg/mL | − | − | − | − |
| during and after | | | | − | − | − | − |
| infection | Dextran | 10 | μg/mL | − | + | ++ | T |
| | (sulphate) | | | − | + | ++ | T |
| | AZT | 0.4 | μm | − | − | − | − |
| | | | | − | − | − | − |

| | | | | |
|---|---|---|---|---|
| Absence of anti-retroviral agent | − | (+) | ++ | T |
| | − | − | − | − |
| | − | (+)/+ | ++ | T |
| | − | −(+)/+ | ++ | T |
| Absence of virus and of anti-retroviral agent | − | − | − | − |
| | − | − | − | − |

The conclusions that can be made in view of the results summarized in Table B are explained in the following.

At day 5, 5 days following infection by the NDK viral strain, the cultures of MT₄ do not form syncitia when they are treated continuously, during and after infection, using PS3 at a concentrations of 10 μg/mL.

However, no inhibitory effect is observed when MT₄ cells are treated only before infection.

In contrast, dextran sulphates has an optimal effect when the cells are treated before viral infection (2 wells out of 2 inhibited).

Furthermore, it appears, and as shown in the experimental series in Table A, that the activity of PS3 is comparable to that of AZT.

The general conclusions stated in the light of the results summarized in Table A apply here.

### EXAMPLE 2B

### Measurement of the Inhibition of RT Activity

In order to evaluate the action of laminarin PS3 and the comparison products on the replication of the infecting virus by measurement of the RT activity in a cell culture, consisting of CEM cells in this experiment, we proceeded as follows.

The number of viruses present in the culture was demonstrated by measuring RT activity in the supernatant of the culture, the RT activity detected quantity being proportional to the number of viruses produced.

The same experimental protocol was used as that in example 2A using MT₄ cells, the difference being that CEM cells were incubated with the sulphated laminarin PS3 or the comparison product and then cultured at a concentration of 0.5*10⁶ cells/mL.

The cells were counted every three days and the cultures diluted with a view of culturing 0.5*10⁶ cells/mL; the RT activity assay was done following a protocol comprising:

the release into culture samples of the viral enzymes, especially RT;

reaction of the samples with a reactive mixture comprising radioactive ³H dTTp (thymidine);

isolation of DNA synthesized as the result of viral replication;

measurement of the radioactivity of the DNA synthesized, said radioactivity being proportional to the quantity of tritiated thymidine incorporated into said synthesized DNA, which itself is proportional to RT activity and thus to the number of viruses produced by replication.

Preparation of the culture samples was done in a P3 protected laboratory.

The contents (1 mL) of each well was centrifuged for 5 minutes at 1500 r.p.m. (Jouan GR 422 centrifuge) and then the culture supernatant obtained was ultracentrifuged at 4° C., 95000 r.p.m. (Beckman TL100 ultracentrifuge) in order to obtain the viral pellet.

The viral pellet was then placed in a test tube containing 10 μL of NTE buffer to which 0.1% of triton was added, which releases the viral enzymes, particularly RT; the tube was then vortexed, covered with parafilm and held for 10 minutes at 4° C. and then frozen at −20° C.

The reaction mixture previously mentioned was prepared in a biochemistry laboratory and this mixture comprises for one 5 mL test tube:

| | |
|---|---|
| base 5X buffer | 10.0 μL |
| Poly rA 1 OD/mL (RNA) | 12.5 μL |
| Oligo dT 1 OD/mL (primer) | 12.5 μL |
| distilled water | 2.5 μL |
| ³H dTTp (thymidine) 1 mCi/mL | 2.5 μL |
| Total | 40.0 μL |

The composition of the aforesaid 5× base buffer is as follows:

| | | Final concentration |
|---|---|---|
| TRIS 1M pH 7.8 | 1.25 mL | 0.25 M |
| MgCl₂ 0.5 M | 1.00 mL | 0.10 M |
| KCl 1 M | 0.50 mL | 0.10 M |
| DTT 100 mM | 0.50 mL | 10 Mm |
| H₂O | 1.75 mL | |

As many test tubes containing this reaction mixture were prepared as there were samples to be tested. In the biochemistry laboratory, these 10 μL samples prepared as described hereinbefore and which contain the virus lysed by the 0.1% triton was introduced into as many tubes, each containing 40 μL of reaction mixture.

These tubes were held for one hour in a warm water bath at 37° C. with agitation every 15 minutes.

The reaction was then stopped by introducing into each tube 1 mL of PPNa (sodium pyrophosphate) 0.1 M prepared in 5% trichloroacetic acid (TCA).

The synthesized DNA contained in the mixture was precipitated at 4° C. by adding 3.5 mL/tube of 20% trichloroacetic acid; it was then filtered over Millipore 0.45μ nitrocellulose filters. To do this, the contents of the tubes were poured into the corresponding wells of a sample collector (Millipore) and then the tubes and the wells were rinsed three times with 5% TCA.

The filters were then dehydrated using 70% alcohol prior to being dried in the over at 80° C. for 20 minutes.

After cooling, the filters were placed individually into vials containing a scintillation or scintillating agent sold under the brand name Emulsifier Safe cat. No. 6013389 (Perkin Elmer).

The count was then done using a liquid scintillation analyzer sold under the name “PACKARD 2100T” and the results expressed in dpm/mL (disintegration per minute and per mL of supernatant).

The amount of radioactivity that was measured is proportional to the RT activity present and, consequently, to the number of viruses produced by replication.

In a first series of experiments, the sulphated laminarin PS3 was tested at concentrations of 5 and 10 μg/mL and the comparison product, AZT, at a concentration of 0.1 μM.

The RTMC virus (clade B) was used for infecting the CEM cells that have the special characteristic of being resistant to AZT and which was used at a dilution of 5*10⁻⁴.

The results recorded on days 3, 7, and 10 are summarized in Table C; these results comprise for each experiment the RT activity expressed in dpm/mL and the numbers of cells are expressed in 10⁶ cells/m L.

TABLE C

| RMTC | | | | Quantity of RT measured and corresponding | | | | | |
|---|---|---|---|---|---|---|---|---|---|
| viral | Anti-retroviral Agent | | Number of cells | number of cells per mL at days 3, 7 and 10 | | | | | |
| dilution | Nature | Concentration | RT quantity | D3 | | D7 | | D10 | |
| 5 · 10⁻⁴ | PS3 | 10 μg/mL | 10⁶ Cell/mL | 0.2 | 3.6 | 0.2 | 0.8 | 1.7 | 3.2 |
| | | | RT (dpm/mL) | 577 | 356 | 1056 | 951 | 3698 | 1737 |
| | | 5 μg/ml | 10⁶ Cell/mL | 3.6 | 4.1 | 1.8 | 1.1 | 1.6 | 0.5 |
| | | | RT (dpm/mL) | 562 | 2439 | 57210 | 2001566 | 150149 | 557329 |
| | AZT | 0.1 μM | 10⁶ Cell/mL | 2.7 | 3.3 | 2.1 | 0.9 | 0.4 | 0.8 |
| | | | RT (dpm/mL) | 639 | 1832 | 37592 | 87550 | 282701 | 1322486 |

| | | | | | | | |
|---|---|---|---|---|---|---|---|
| Absence of anti- | 10⁶ Cell/mL | 4 | 5.7 | 1 | 0.6 | 0.6 | 0.6 |
| retroviral agent | RT (dpm/mL) | 4905 | 6393 | 1347767 | 1274569 | 1152701 | 52907 |
| Absence of virus | 10⁶ Cell/mL | 3.9 | 5 | 1.6 | 2.6 | 7 | 4.6 |
| Absence of anti- | RT (dpm/mL) | 245 | 782 | 1156 | 2090 | 1685 | 1195 |
| retroviral agent | | | | | | | |

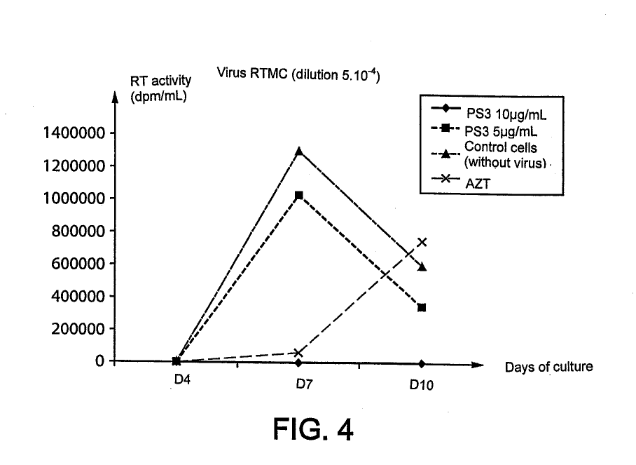

To better demonstrate the action of the sulphated laminarin PS3 the RT activity evolution expressed in dpm/mL as a function of time (days 3 to 10) has been transposed to the graph of FIG. 4 using the results summarized in Table C, for the cells treated at two concentrations of PS3 and with AZT, respectively, as well as for the control cells (no agent) using a viral dilution of 5*10⁻⁴.

An examination of Table C and FIG. 4 allows the conclusion that at the concentration of 10 μg/mL, PS3 provides inhibition of the RMTC virus approaching 100% at a viral concentration of 5*10⁻⁴.

It was indeed demonstrated the PS3 is effective on the RMTC virus which is resistant to ATZ, a well known inhibitor of RT.

### EXAMPLE 3

### Study of the Action of Sulphated PS3 as a Function of the Moment of its Incorporation

The action of sulphated laminarin PS3 as a function of its moment of incorporation (continuous, before infection, during infection and after infection) was studied.

The procedure was the same as that previously indicated with regard to the experiments carried out on MT₄ cells; e.g., as in Example 2A.

Again, the PS3 was tested at concentrations of 5 and 10 μg/mL and AZT at a concentration of 0.1 μM and this time the virus used was the NKD (clade D) virus at a dilution of 2.5*10⁻⁵.

The results of the measurements done on days 3 and 7 are summarized in Table D; RT activity is expressed in dpm/mL and cells are expressed in 10⁶ cells/mL.

TABLE D

| | | | | Quantity of RT measured and | | | |
|---|---|---|---|---|---|---|---|
| | | | | corresponding number of cells at | | | |
| Anti-retroviral Agent | | | Number of cells | days 3, 7 and 10 | | | |
| Incorporation | Nature | Concentration | RT quantity | J3 | | J7 | |
| Continously, | PS3 | 10 μg/mL | 10⁶ Cells/mL | 1.6 | 1.5 | 3.1 | 3.6 |
| before, during | | | RT (dpm/mL) | 471 | 603 | 1307 | 88808 |
| and after | | 5 μg/mL | 10⁶ Cells/mL | 1.1 | 1.6 | 3.3 | 3.4 |
| | | | RT (dpm/mL) | 377 | 469 | 926 | 715 |
| | AZT | 0.1 μM | 10⁶ Cells/mL | 1.7 | 1.3 | 3.6 | 3.2 |
| | | | RT (dpm/mL) | 399 | 750 | 14988 | 48013 |
| Before infection | PS3 | 10 μg/mL | 10⁶ Cells/mL | 0.9 | 2 | 1.4 | 1.7 |
| | | | RT (dpm/mL) | 1321 | 2199 | 1543435 | 1480097 |
| | | 5 μg/mL | 10⁶ Cells/mL | 1.2 | 1.3 | 2.5 | 2.4 |
| | | | RT (dpm/mL) | 713 | 245 | 285150 | 505929 |
| | AZT | 0.1 μM | 10⁶ Cells/mL | 1.4 | 0.8 | 2 | 3.8 |
| | | | RT (dpm/mL) | 1251 | 529 | 1381090 | 673 |
| During dining | PS3 | 10 μg/mL | 10⁶ Cells/mL | 1.3 | 1.4 | 2.5 | 2.4 |
| room | | | RT (dpm/mL) | 571 | 930 | 50601 | 1106 |
| | | 5 μg/mL | 10⁶ Cells/mL | 1.2 | 1.5 | 2 | 2.1 |
| | | | RT (dpm/mL) | 756 | 761 | 1298585 | 1753853 |
| | AZT | 0.1 μM | 10⁶ Cells/mL | 1.9 | 1.6 | 3.2 | 1.6 |
| | | | RT (dpm/mL) | 1529 | 956 | 94848 | 1259346 |
| After infection | PS3 | 10 μg/mL | 10⁶ Cells/mL | 1.7 | 1.3 | 2.6 | 2.4 |
| | | | RT (dpm/mL) | 441 | 741 | 1665 | 830254 |
| | | 5 μg/mL | 10⁶ Cells/mL | 1.1 | 1 | 2.6 | 2.6 |
| | | | RT (dpm/mL) | 1121 | 658 | 460595 | 1502670 |
| | AZT | 0.1 μM | 10⁶ Cells/mL | 1.2 | 0.8 | 1.8 | 3.2 |
| | | | RT (dpm/mL) | 1249 | 956 | 3096138 | 1098 |

| | | | | | |
|---|---|---|---|---|---|
| Absence of anti-retroviral agent | 10⁶ Cells/mL | 0.8 | 0.8 | 2.7 | 2.6 |
| | RT (dpm/mL) | 341 | 241 | 1653978 | 905197 |
| Absence of virus and of anti-retroviral | 10⁶ Cells/mL | 0.6 | 0.9 | 3.2 | 3.6 |
| agent | RT (dpm/mL) | 231 | 418 | 1362 | 351 |

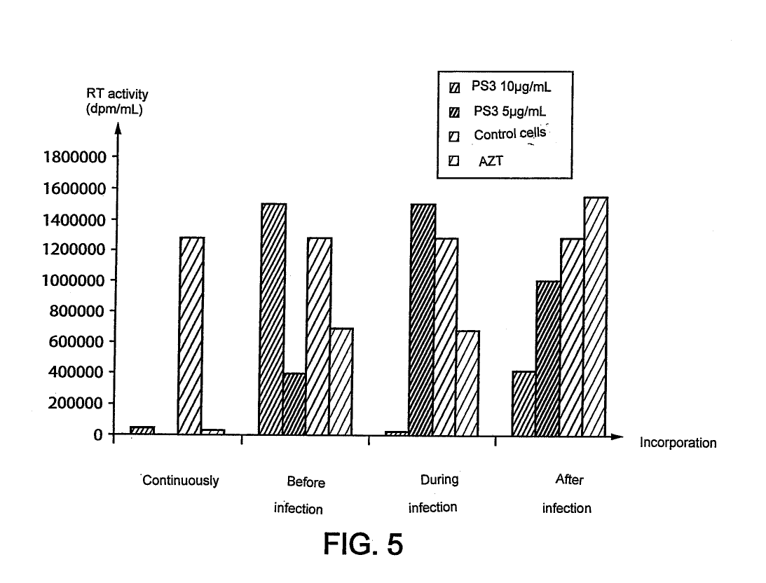

In FIG. 5, which illustrates Table D, a graphic is used to show the RT activity expressed in dpm/mL measured at day 7 in the case of AZT and of two concentrations of PS3 as well as the case of the control cells that were not treated with an anti-retroviral agent and represents the four experiments corresponding to the administration of the anti-retroviral agent continuously, before, during and after infection with the NDK virus at a dilution of 2.5*10⁻⁵.

On examination of Table D and FIG. 5 it appears that PS3 at the concentration of 10 μg/mL has no action when it is incubated before infection, that it is most efficient when it is present throughout the experiment and that it has action when it is incorporated during and after infection.

At all times at the concentration of 10 μg/mL, its greater activity with respect to AZT is clearly apparent.

### EXAMPLE 4

### Direct Inhibitory Action of PS3

A certain number of supplementary experiments were also done in order to study the direct inhibitory action of PS3 on the RT of the RW92009, UG92020P, PIC CH and NDK viruses.

To do this, the RT activity of the four strains of the virus that were identified and placed in contact with PS3 as well as in certain cases with dextran sulphate, used as a comparison product, was measured.

In order to perform these measurements, the method described in detail hereinbefore was followed (Example 2).

The results recorded at the end of said measurements is shown in the graphs that are illustrated in FIGS. 6 to 9, wherein the ordinate axis corresponds to the percent of inhibition of RT activity (this activity is expressed in dpm/mL) measured for different concentrations of PS3 or of dextran sulphate which are indicated on the abscissa axis, these concentrations being expressed in μg/mL.

For the RW92009 virus, a viral concentration corresponding to an RT activity of 75,000 dpm was used; the results obtained appear in the graph in FIG. 6, wherein it is seen that PS3 at a concentration of 1.5 μg/mL induces a 45% inhibition of RT.

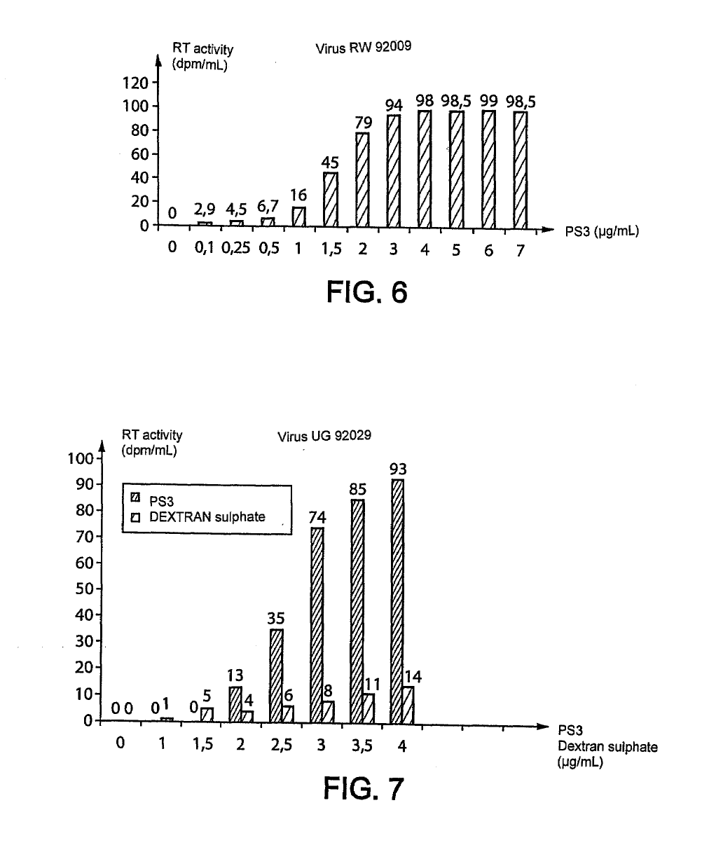

For the UG92029 virus, a viral concentration corresponding to an RT activity of 220,000 dpm was used; the results obtained appear in the graph in FIG. 7 that shows that PS3 at a concentration of 2.5 μg/mL induces a 35% inhibition of RT, while dextran sulphate inhibits only 6% of the RT activity at the same concentration.

In addition, it is noted that at the concentration of 3 μg/mL there is a 74% inhibition for PS3 compared to 8% for dextran sulphate.

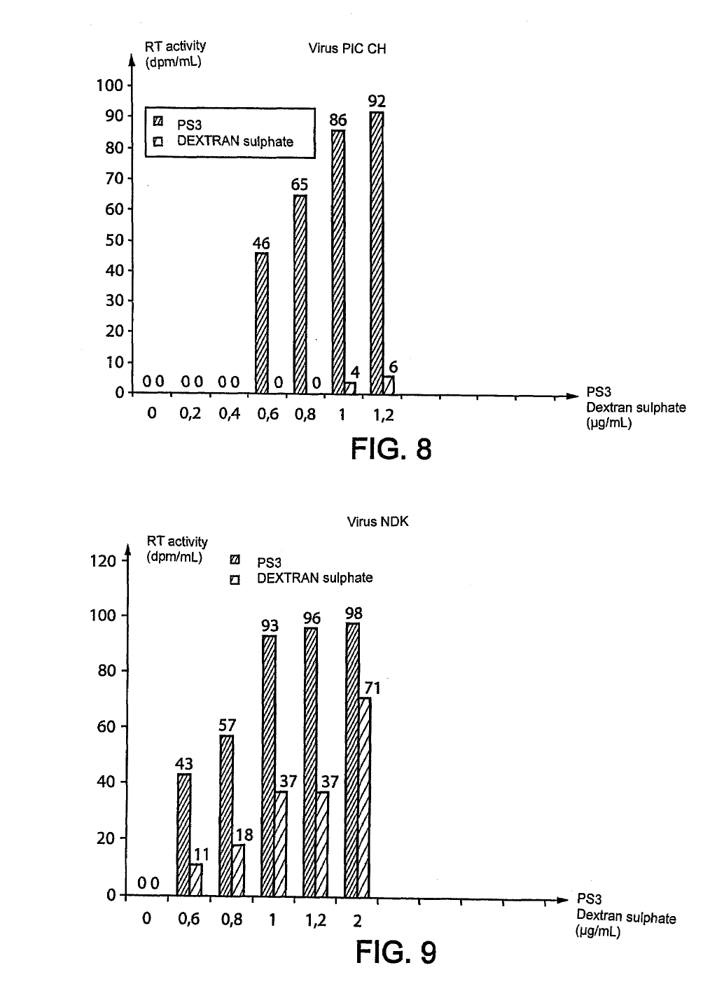

For the PIC CH virus, a viral concentration corresponding to a RT activity of 105,000 dpm was used; the results obtained are shown in the graph of FIG. 8; examination of this graph confirms that PS3 at a concentration of 0.6 μg/mL induces a 46% inhibition of RT, while dextran sulphate does not produce any inhibition at all at the same concentration; at the concentration of 0.8 μg/mL, 65% inhibition is obtained in the case of PS3, while dextran sulphate still does not induce any inhibition.

For the NDK virus a viral concentration corresponding to an RT activity of 81,000 dpm was used; the results obtained appear in the graph in FIG. 9, which shows that at the concentration of 0.6 μg/mL PS3 induces a 43% inhibition of RT compared to only 11% in the case of dextran sulphate; at the concentration of 0.8 μg/mL a 57% inhibition is obtained in the case of PS3 compared to 18% in the case of dextran sulphate.

The results of the experiments shown in FIGS. 6 to 9 make it possible to confirm that PS3 is an inhibitor of RT in different strains of HIV, which indicates early inhibition of the replication cycle of these strains.

PS3 is particularly efficient on the RT of the PIC CH (clade B), which is resistant to several antiviral agents as well as on the NDK (clade D) virus.

### EXAMPLE 5

### Measurement of the Anti-Coagulant Activity of Sulphated Laminarin PS3

It was shown that the anticoagulant activity of sulphated laminarin PS3 is sufficiently low compared to that of heparin and does not constitute a drawback in the context of its use according to the invention and it is not cytotoxic at the maximum concentrations likely of being used.

Accordingly, the anticoagulant activity of sulphated laminarin PS3 obtained in Example 1 was determined as a function of its concentration in comparison to that of heparin in classical APTT (partial activated thromboplastin time) coagulation tests, the prothrombin time, the HEPTEST, and the thrombin time. The APTT reflects an interaction with the intrinsic coagulation system while the prothrombin time reflects an interaction with the extrinsic coagulation system; the so-called HEPTEST is the classical test for measuring the inhibitory activity of heparin with regard to Factor Xa, and the thrombin time corresponds to the last step of coagulation, e.g. the formation of fibrin induced by thrombin. It was found that in contrast with that of heparin, the activity of sulphated laminarin PS3 in the so-called HEPTEST is more than 20 times lower. Likewise, as concerns the prothrombin time sulphated laminarin PS3 does not exhibit any pronounced anticoagulant effect as in the case of heparin. The specific activity (IU/mg) in the APPT represents 30% of the activity of heparin and in the case of the thrombin time, 60%. In order to completely prevent coagulation, in the APTT a concentration of 4-times higher and in the case of the thrombin time a concentration 20-times higher had to be administered.

In the specific anti-Factor Xa and the anti-thrombin tests using chromogenic substances it was found that sulphated laminarin PS3, in contrast with heparin, does not have noteworthy anti-Factor Xa activity depending on anti-thrombin or anti-thrombin activity. The effect in the case of the thrombin time can be considered as being due to an inhibition of thrombin dependent on the heparin cofactor II. By reason on the one hand of the lower specific activity and on the other hand on the concentration-dependent profile and still other studies relating to the mechanism of action, it is possible to consider that in the case of sulphated laminarin PS3 the bleeding risk is substantially lower than in the case of heparin.

It follows that the RT inhibitory properties of sulphated laminarin PS3 could be used advantageously without fear of undesirable secondary effects on coagulation.

### EXAMPLE 6

### Measurement of the In Vitro Cytotoxicity of Sulphated Laminarin PS3

The in vitro toxicity of sulphated laminarin PS3 obtained in Example 1 was determined simultaneously with that of a comparison product.

To do this, CEM cells were cultivated in a 24-well plate in 1 mL of RPMI supplemented with 10% foetal calf serum, 1% penicillin-streptomycin, 2 mM of glutamine, 2 μg/mL of polybrene and different concentrations of sulphated laminarin PS3 and comparison product comprised of dextran sulphate.

Four different concentrations of sulphated laminarin PS3 were tested: 125 μg/mL, 250 μg/mL, 500 μg/mL, and 1000 μg/mL.

The lowest of these concentration is already greater than the concentration of 10 μg/mL whose efficacy was shown by the previous experiments.

All of the tests were done in duplicate.

The cells were counted every day and their numeric increase was compare to that of a control culture comprised of CEM cells cultured in the absence of sulphated laminarin PS3 or dextran sulphate (0 μg/mL).

Table E summarizes the results recorded at the first, second and third day of the experiment; in other words, the number of cells counted in each culture.

In the light of the results shown in Table E, it would appear that PS3 does not exhibit cytotoxicity unless in very high concentrations, in fact at 1 mg/m L.

TABLE E

| | Number of CEM | |
|---|---|---|
| | cells (×10⁶/mL) | |
| | in the presence of PS3 or | |
| | dextran sulphate | |
| Concentration (μg/mL) | PS3 | Dextran sulphate |

Day 1

| | | | | |
|---|---|---|---|---|
| 1000 | 0.8 | 0.5 | 0.8 | 0.5 |
| 500 | 1 | 0.7 | 1 | 0.3 |
| 250 | 0.7 | 0.7 | 0.5 | 0.9 |
| 125 | 0.6 | 0.5 | 0.5 | 0.9 |
| Control - absence of anti- | 0.9 | 0.4 | 0.9 | 0.4 |
| retroviral agent | | | | |

Day 2

| | | | | |
|---|---|---|---|---|
| 1000 | 1.3 | 1 | 1.3 | 1.7 |
| 500 | 1.4 | 1.5 | 1.3 | 1.7 |
| 250 | 1.2 | 1.4 | 1.3 | 1.4 |
| 125 | 0.9 | 1.1 | 1.5 | 1 |
| Control - absence of anti- | 1.3 | 1.2 | 1.3 | 1.2 |
| retroviral agent | | | | |

Day 3

| | | | | |
|---|---|---|---|---|
| 1000 | 2.4 | 2.8 | 2 | 2.7 |
| 500 | 3.2 | 2.3 | 2.1 | 1.5 |
| 250 | 2.4 | 2.2 | 1.9 | 1.9 |
| 125 | 2.4 | 2.3 | 2.3 | 2.1 |
| Control - absence of anti- | 2.4 | 2 | 2.4 | 2 |
| retroviral agent | | | | |

### EXAMPLE 7

### Measurement of the In Vivo Cytotoxicity of Sulphated Laminarin PS3

Possible in vivo toxicity of sulphated laminarin PS3 used according to the invention was studied.

This study was done on the New Zealand white rabbits and on Sprague Dawley rats.

The white rabbits were subjected on the one hand to ocular irritation test and on the other hand to the primary cutaneous irritation test.

The outcome of the first of these tests allows the conclusion of a mildly irritant action and in the second test non-irritant action.

Then the rats were subjected on the one hand to a study for determining acute dermal toxicity, and on the other hand to a study for determining acute oral toxicity.

In the first test, the lethal dermal dose 50 (LDD₅₀) is greater than 2 g/kg BW (Body Weight) that allows the conclusion that the product is not toxic.

In the second test, the acute toxicity by oral route can be considered to be greater than 2 g/kg BW, which allows, again, the product to be classified as non-toxic.

The experiments that allowed these conclusions were done under the direction of Dr R. SHRIVASTAVA in the Department of Toxicology at the facility called

### Elevage Scientifique des Dombes (ESD)

### ROMANS

### 01400 CHATILLON SUR CHALARONNE (FRANCE)

in compliance with the guidelines of the OCDE No. 404 and 405 of Feb. 24, 1987, as relates to the studies done on white rabbits and the governing provisions of the OCDE No. 401 and 402 (1987) as well as EEC directive B-1 92/69 (1992) as relates to the studies done on the Sprague Dawley rats.

### EXAMPLE 8

### Composition of a Sulphated Laminarin PS3 Based Cream

A sulphated laminarin PS3 based cream was made having the following composition:

| | |
|---|---|
| Demineralised water | 69.7% |
| Glycerine | 5.0% |
| sulphated laminarin PS3 | 1.0% |
| PEG 100 stearate | 4.0% |
| Cetearylic alcohol | 2.0% |
| Preservative | 1.0% |
| PEG 40 stearate | 3.0% |
| Vitamin E acetate | 0.5% |
| C - 12-15 alkyl benzoate | 6.5% |
| Caprylic/capric triglycerides | 5.5% |
| NaOH 0.1 N | 1.8% |
| | 100% |

It is possible to provide 2 to 5 applications per day.

### EXAMPLE 9

### Composition of a Sulphated Laminarin PS3 Based Aerosol Solution

A sulphated laminarin PS3 based solution for aerosol use was made having the following composition:

| | |
|---|---|
| Sulphated laminarin PS3 | 2.5% |
| Sodium chloride | 9.0% |
| Demineralised water Eu. Pharm. | 88.5% |

It is possible to administer a daily quantity of the aerosol equivalent to a quantity of 1000 to 10,000 μg of active substance.

### EXAMPLE 10

### Composition of a β 1-3 Glucan Based Suppository

A β 1-3 glucan-based suppository was made having the following composition:

| | |
|---|---|
| β 1-3 glucan sulphate potassium salt | 5.0% |
| (DP = 22-24, sulphation degree 2.4) | |
| Solid semi-synthetic glycerines | 95.0% |

It is recommended that 1 or 2 be administered per day.

### EXAMPLE 11

### Composition a Laminaritol Sulphate Based Injectible Solution

A laminaritol sulphate based solution for injection was made having the following composition:

| | |
|---|---|
| Laminaritol sulphate sodium salt | 5.0% |
| (DP = 20, sulphation degree 2.4) | |
| Sodium bicarbonate | 3.0% |
| ppi water | 92.0% |

It is possible to administer from 1000 to 3000 mL of the injectible solution over a period of 24 hours.

### EXAMPLE 12

### Composition of a β 1-3 Glucan Sulphate Based Vaginal Solution

A β 1-3 glucan sulphate based vaginal solution was made having the following composition:

| | |
|---|---|
| β 1-3 glucan sulphate sodium salt | 0.1% |
| (DP = 20 sulphation degree 2.4) | |
| Sodium chloride | 9.0% |
| Ethyl alcohol 95° | 5.0% |
| Rose fragrance | 0.2% |
| Purified water | 84.7% |
| Preservatives (bezalkonium chloride) | 0.2% |
| Sodium edatate | 0.3% |
| Polysorbate 20 | 0.5% |
| | 100% |

It is possible to make one or two applications per day.

Considering the whole set of experimental results described in the aforegoing, it is possible to conclude that the polysaccharides of formula (I) and more particularly sulphated laminarin have significant anti-retroviral activity, especially on the replication cycle of the HIV.

The moment of administration at which sulphated laminarin PS3 is efficient shows a specific action on the early events of the viral replication cycle.

No cellular toxicity was observed at the inhibitory doses tested or at 1 mg/mL.

Furthermore, the absence of toxicity of laminarin sulphate, even at 1 mg/mL, on CEM cells is evidence of a therapeutic index of greater than 200.

The anti-retroviral activity of the polysaccharides of formula (I), particularly that of sulphated laminarin, is not only better than that of the products previously used but, moreover, it acts even on the viruses that are resistant to some known RT inhibitors, e.g. the PIC CH virus that is resistant to d4T and to Zerit and the RTMC virus that is resistant to AZT.

The polysaccharides of formula (I), and most particularly sulphated laminarin, inhibit the RT of the isolated viruses, which appears to eliminate the hypothesis of a mechanism of action linked to the simple anionic character of the sulphated polysaccharides of the invention.

1. Use of a polysaccharide having the formula (1) 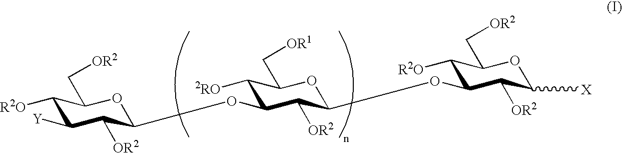
wherein
R¹ represents either a hydrogen atom, a sulphate group or a phosphate group or a sulphated or phosphated glucose preferably linked by a β(1→6) type link to the saccharide structure,
R² represents a hydrogen atom, a sulphate group or a phosphate group, provided that R¹ and R² do not represent simultaneously a hydrogen atom,
X and Y represent, each independently, an OH group, a glucose, a sulphated or phosphated glucose, a mannitol or a sulphated or phosphated mannitol,
n represents an integer from 11 to 30, preferably from 20 to 30 and more preferably from 25 to 30,
said polysaccharide having a sulphation degree greater than 2, preferably from 2.2 to 2.4, or a phosphation degree greater than 1, preferably 1.5 to 2.5, for the manufacture of a medicament for the treatment of retroviral diseases.

2. Use of a polysaccharide having the formula (1) 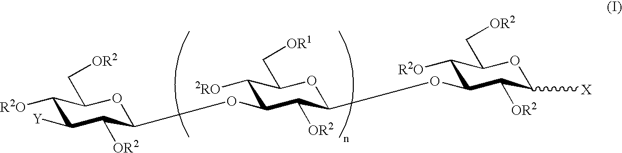
wherein
R¹ represents either a hydrogen atom, a sulphate group or a phosphate group or a sulphated or phosphated glucose preferably linked by a β(1→6) type link to the saccharide structure,
R² represents a hydrogen atom, a sulphate group or a phosphate group, provided that R¹ and R² do not represent simultaneously a hydrogen atom,
X and Y represent, each independently, an OH group, a glucose, a sulphated or phosphated glucose, a mannitol or a sulphated or phosphated mannitol,
n represents an integer from 11 to 30, preferably from 20 to 30 and more preferably from 25 to 30,
said polysaccharide having a sulphation degree greater than 2, preferably from 2.2 to 2.4, or a phosphation degree greater than 1, preferably 1.5 to 2.5, for implementing a method of treatment of retroviral diseases.

3. Use according to claim 1, wherein the polysaccharide of formula (I) is a sulphated laminarin having a polymerisation degree of 11 to 28.

4. Use according to claim 1, wherein the polysaccharide of formula (I) is a phosphated laminarin having a polymerisation degree of 11 to 28.

5. Use according to claim 1, wherein the retroviral diseases are preferentially selected from among those caused by the lentiviruses and the oncoviruses, more particularly by the HIV and by strains of these retroviruses that are resistant to already known anti-retroviral agents.

6. Use according to claim 1, wherein the retroviral disease is the acquired immunodeficiency syndrome or AIDS in the human being.

7. Use according to claim 1, wherein the retroviral diseases are the cancers associated with the oncoviruses.

8. A combination product comprising an effective amount
a polysaccharide having the formula (I) wherein R₁ represents either a hydrogen atom, a sulphate group or a phosphate group or a sulphated or phosphated glucose preferably linked by a β(1→6) type linkage to the saccharide structure, R₂ represents a hydrogen atom, a sulphate group or a phosphate group, X and Y each independently represent an OH group, a glucose, a sulphated or phosphated glucose, a mannitol or a sulphated or phosphated mannitol, n represents an integer from 11 to 30, preferably from 20 to 30, more preferably from 25 to 30, said polysaccharide having a sulphation degree greater than 2, preferably from 2.2 to 2.4, or a phosphation degree greater than 1, preferably from 1.5 to 2.5,
of at least one anti-retroviral agent selected from the group comprising:
nucleoside inhibitors of reverse transcriptase (NIRT), notably AZT, ddl, ddC, d4T, 3TC and ABC,
the non-nucleoside inhibitors of reverse transcriptase (NNIRT), notably Viramune and Sustiva,
the protease inhibitors, notably Agnerase and Kaletra,
the fusion inhibitors, notably enfuvirtide (Fuzeon),
the entry inhibitors, notably AMD-3100 and, optionally
at least one pharmacological agent selected from the group comprising the anti-nausea agents, the anti-diarrhoea agents, the anti-hyperbilirubinemia agents, the analgesic agents, the dermatological treatment agents, the anti-nephrotoxic agents, for simultaneous, separate or stepped use over time.

9. Use of an effective amount of
a polysaccharide having the formula (I) wherein R₁ represents either a hydrogen atom, a sulphate group or a phosphate group or a sulphated or phosphated glucose preferably linked by a β(1→6) type linkage to the saccharide structure, R₂ represents a hydrogen atom, a sulphate group or a phosphate group, X and Y each independently represent an OH group, a glucose, a sulphated or phosphated glucose, a mannitol or a sulphated or phosphated mannitol, n represents an integer from 11 to 30, preferably from 20 to 30, more preferably from 25 to 30, said polysaccharide having a sulphation degree greater than 2, preferably from 2.2 to 2.4, or a phosphation degree greater than 1, preferably from 1.5 to 2.5,
of at least one anti-retroviral agent selected from the group comprising:
nucleoside inhibitors of reverse transcriptase (NIRT), notably AZT, ddl, ddC, d4T, 3TC and ABC,
the non-nucleoside inhibitors of reverse transcriptase (NNIRT), notably Viramune and Sustiva,
the protease inhibitors, notably Agnerase and Kaletra,
the fusion inhibitors, notably enfuvirtide (Fuzeon),
the entry inhibitors, notably AMD-3100 and, optionally
at least one pharmacological agent selected from the group comprising the anti-nausea agents, the anti-diarrhoea agents, the anti-hyperbilirubinemia agents, the analgesic agents, the dermatological treatment agents, the anti-nephrotoxic agents, for the manufacture of a medicament for the treatment of retroviral diseases, preferably those caused by the lentiviruses and the oncoviruses, more preferably caused by HIV, particularly by the strains of these viruses that are resistant to the already known anti-retroviral agents.

10. Use of an effective amount of
a polysaccharide having the formula (I) wherein R₁ represents either a hydrogen atom, a sulphate group or a phosphate group or a sulphated or phosphated glucose preferably linked by a β(1→6) type linkage to the saccharide structure, R₂ represents a hydrogen atom, a sulphate group or a phosphate group, X and Y each independently represent an OH group, a glucose, a sulphated or phosphated glucose, a mannitol or a sulphated or phosphated mannitol, n represents an integer from 11 to 30, preferably from 20 to 30, more preferably from 25 to 30, said polysaccharide having a sulphation degree greater than 2, preferably from 2.2 to 2.4, or a phosphation degree greater than 1, preferably from 1.5 to 2.5,
of at least one anti-retroviral agent selected from the group comprising:
nucleoside inhibitors of reverse transcriptase (NIRT), notably AZT, ddl, ddC, d4T, 3TC and ABC,
the non-nucleoside inhibitors of reverse transcriptase (NNIRT), notably Viramune and Sustiva,
the protease inhibitors, notably Agnerase and Kaletra,
the fusion inhibitors, notably enfuvirtide (Fuzeon),
the entry inhibitors, notably AMD-3100 and, optionally
at least one pharmacological agent selected from the group comprising the anti-nausea agents, the anti-diarrhoea agents, the anti-hyperbilirubinemia agents, the analgesic agents, the dermatological treatment agents, the anti-nephrotoxic agents, for the implementation of a method of treatment of retroviral diseases, preferably those caused by the lentiviruses and the oncoviruses, more preferably caused by HIV, particularly by the strains of these viruses that are resistant to the already known anti-retroviral agents.

11. A method of treatment of a retroviral disease, preferably caused by the lentiviruses and the oncoviruses, more preferably by HIV, especially by the strains of these retroviruses that are resistant to the already known anti-retroviral agents, comprising the administration to a patient with said retroviral disease of an effective amount of a medicament comprising as its active agent at least one polysaccharide having formula (I) 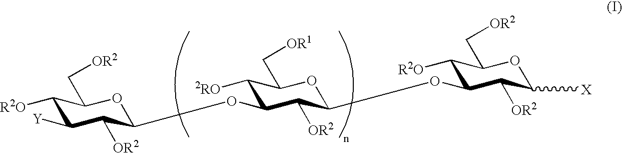
wherein R₁ represents either a hydrogen atom, a sulphate group or a phosphate group, or a sulphated or phosphated glucose preferably linked by a β(1→6) type linkage to the saccharide structure, R₂ represents a hydrogen atom, a sulphate group or a phosphate group, X and Y each independently represent an OH group, a glucose, a sulphated or phosphated glucose, a mannitol, or a sulphated or phosphated mannitol, n represents an integer from 11 to 30, preferably from 20 to 30, more preferably from 25 to 30, said polysaccharide having a sulphation degree greater than 2, preferably from 2.2 to 2.4, or a phosphation degree greater than 1, preferably from 1.5 to 2.5; or of a combination product as claimed in claim 8.

12. Method of treatment according to claim 11, wherein the polysaccharide having formula (I) is a sulphated laminarin having a sulphation degree greater than 2, preferably 2.2. to 2.4, and a polymerisation degree of 11 to 28.

13. Method of treatment according to claim 11, wherein the polysaccharide having formula (I) is a phosphated laminarin having a phosphation degree greater than 1, preferably 1.5. to 2.5, and a polymerisation degree of 11 to 28.

14. Use according to claim 2, wherein the polysaccharide of formula (I) is a sulphated laminarin having a polymerisation degree of 11 to 28.

15. Use according to claim 2, wherein the polysaccharide of formula (I) is a phosphated laminarin having a polymerisation degree of 11 to 28.

16. Use according to claim 2, wherein the retroviral diseases are preferentially selected from among those caused by the lentiviruses and the oncoviruses, more particularly by the HIV and by strains of these retroviruses that are resistant to already known anti-retroviral agents.

17. Use according to claim 2, wherein the retroviral disease is the acquired immunodeficiency syndrome or AIDS in the human being.

18. Use according to claim 2, wherein the retroviral diseases are the cancers associated with the oncoviruses.
